(19) United States
(12) Patent Application Publication
(10) Pub. No.: US 2019/0202940 A1
(43) Pub. Date: Jul. 4, 2019

(54) METHOD FOR PREPARING CELLULOSE-CONTAINING PARTICLES

(71) Applicant: CELLUCOMP LIMITED, Bumtisland Fife (GB)

(72) Inventors: David HEPWORTH, Burntisland Fife (GB); Eric WHALE, Burntisland Fife (GB)

(21) Appl. No.: 16/311,725

(22) PCT Filed: Jun. 23, 2017
(86) PCT No.: PCT/EP2017/065532

§ 371 Date: Dec. 20, 2018

(30) Foreign Application Priority Data
Jun. 23, 2016 (GB) 1610961.3

(51) Int. Cl. C08B 15/00; C08L 1/02; C09D 101/02; A61K 8/73; C09K 8/20; A23L 29/262; A23L 2/52

(52) U.S. Cl. CPC C08B 15/00; C08L 1/02; C09D 101/02; A23V 2002/00; C09K 8/206; A23L 29/262; A23L 2/52; A61K 8/731

(57) ABSTRACT

There is described a novel method for processing plant material to produce a cellulose-containing material. The process comprises the steps of: (i) contacting particles of plant material with a peroxide agent and water; (ii) allowing the mixture from step (i) to hydrate until the pH of the mixture is pH 4.5 or less; and (iii) homogenising the mixture from step (ii) and isolating the cellulose-containing-material, and wherein the particles of plant material in step (i) have an average particle diameter of from 10 μm to 800 μm.

### TECHNICAL FIELD

The present invention relates to a process for preparing cellulose-containing Particles from plant material with improved efficiency. In particular, the process can be operated to permit continuous manufacture of the cellulose-containing particles. The cellulose-containing Particles are useful as rheology modifiers and anti-cracking agents in a wide range of products.

### BACKGROUND TO THE INVENTION

The present invention relates generally to the field of cellulosic processing, more specifically to the processing of cellulose into a form which can be useful in a wide range of applications. Cellulose is known to exhibit desirable properties in terms of its strength, biodegradability and reinforcing properties. Micro-fibrillated cellulose (MFC) and nano-fibrillated cellulose (NFC) are both of particular importance in this regard, and much effort has been expended in developing suitable processes for their production, and for developing other useful cellulose products.

Prior art processes for the production of cellulose commence with plant material (more frequently wood or cotton waste) which is then pulped and processed to extract the cellulose. Some processes rely upon mechanical processing alone, whilst other processes use a combination of chemical and/or enzymatic treatment, together with mechanical processing. For example, US 2015/0337493 describes extraction of cellulose by mechanically fibrillating a pulp, and US 2005/0274469 suggests application of high shear to pre-soaked cellulosic material followed by fluid bed drying or flash drying. Prior art processes incorporating chemical/enzyme treatment(s) include US 2006/0289132 which describes the use of an oxidant and a transition metal in an aqueous suspension of the starting material pulp followed by mechanical delamination. WO 2015/007953 suggests adding an oxidant to an aqueous pulp suspension followed by mechanical mixing or shearing of the suspension. WO 2013/188657 describes treating an aqueous slurry of starting material with an ozone and/or cellulase whilst concurrently or subsequently comminuting the material. In WO 2014/147392 and WO 2014/147393, CelluComp Ltd refer to a method of processing cellulose-containing compositions from herbaceous plant material in a manner which produces a cellulose end product of a required viscosity with reduced wastage. However, the method requires processing a pulp of high viscosity so that the process needs to be conducted at low solids levels. U.S. Pat. No. 5,964,983 also refers to a process for preparing cellulose from herbaceous plant material using acid or basic hydrolysis. U.S. Pat. No. 5,965,4983 suggests that the dehydrated pulp can be ground to reduce abrasive calcium oxalate crystals which affect homogenization.

CN104963026 discloses treating a plant material from wormwood with hydrogen peroxide in the manufacture of viscose rayon fibre. CN102020723 discloses homogenizing a plant material from the tuber Jerusalem artichoke and then bleaching it with hydrogen peroxide to form an aqueous mixture of pH 5-7. GB577562 discloses reducing the viscosity of wood pulp by treating it with hydrogen peroxide to form an aqueous mixture of pH 6-8. U.S. Pat. No. 6,083,582 discloses peroxide bleaching of a non-wood plant material and then homogenizing the resulting mixture.

One recognised problem with known cellulose processes is that such processes often require high levels of energy. The cost of providing the energy needed frequently renders the process uneconomical to operate at a commercial level.

A further problem is that the starting material is processed in the form of a pulp, so that the process requires considerable quantities of water. In addition to the environmental consideration of high water usage, further cost is added due to the necessity of treating the waste water to remove contaminants. Also, as the plant material is processed, the plant wall deteriorates to release cellulose which causes the viscosity of the pulp to increase considerably. Consequently, the processes of the prior art are typically conducted at very low concentrations of solids, again adding to the difficulties of producing the required cellulose end product in a cost-effective and efficient manner. Particular difficulties are experienced in washing and filtering the high viscosity product. Additionally, due to the viscosity of the slurry, many of the prior art processes can only be operated batch-wise, rather than in a continuous manner.

### SUMMARY OF THE INVENTION

In a first aspect, the present invention provides a process for preparing cellulose-containing material, the process comprising the steps of:

(i) contacting particles of plant material with a peroxide agent and water;
(ii) allowing the mixture from step (i) to hydrate until the pH of the mixture is pH 4.5 or lower; and
(iii) homogenising the mixture from step (ii) and isolating the cellulose-containing material, and wherein the particles of plant material in step (i) have an average particle diameter of from 10 μm to 800 μm.

In a second aspect, the present invention provides a process for preparing cellulose-containing material, the process comprising the steps of: (a) comminuting plant material to form particles of plant material, said particles having an average particle diameter of from 10 μm to 800 μm; (b)
(i) contacting the particles of plant material with a peroxide reagent and water;
(ii) allowing the mixture from step (i) to hydrate until the pH of the mixture is pH 4.5 or lower;
(iii) homogenising the mixture from step (ii) and isolating the cellulose-containing material.

Surprisingly, the present process finds several unexpected advantages despite commencing with particles of plant material of a size broadly equivalent to that obtained in prior art processes which homogenise plant material in water to form a slurry. The advantages noted include a viscosity for the present slurry obtained in step (i) which allows improved processing at a higher solids content relative to prior art processes. Also, the obtained products show surprising benefits when formulating among others into paint and plaster compositions.

It has surprisingly been found that forming the plant material into the particles prior to processing to degrade the cell wall enables the viscosity of the mixture to be kept to a level which facilitates processing steps such as reagent addition, heating and washing. The advantages of the invention are obtained when the mean average particle diameter is from 10 to 800 μm.

Although we do not wish to be bound by theory, it is hypothesised that particles below a mean average diameter of 10 μm produce an inferior end product as the chemical reactions progress too quickly and cellulose is degraded too far. In contrast, particles having a diameter of over 800 μm react unevenly, with the core of the particles being essentially under reacted, again leading to inferior product.

In a third aspect, the present invention provides cellulose-containing material obtainable by the process of the present invention.

### BRIEF DESCRIPTION OF THE FIGURES

FIG. 1 shows the particle size distribution of the powder used within the examples and the fractions which are separated by sifting.

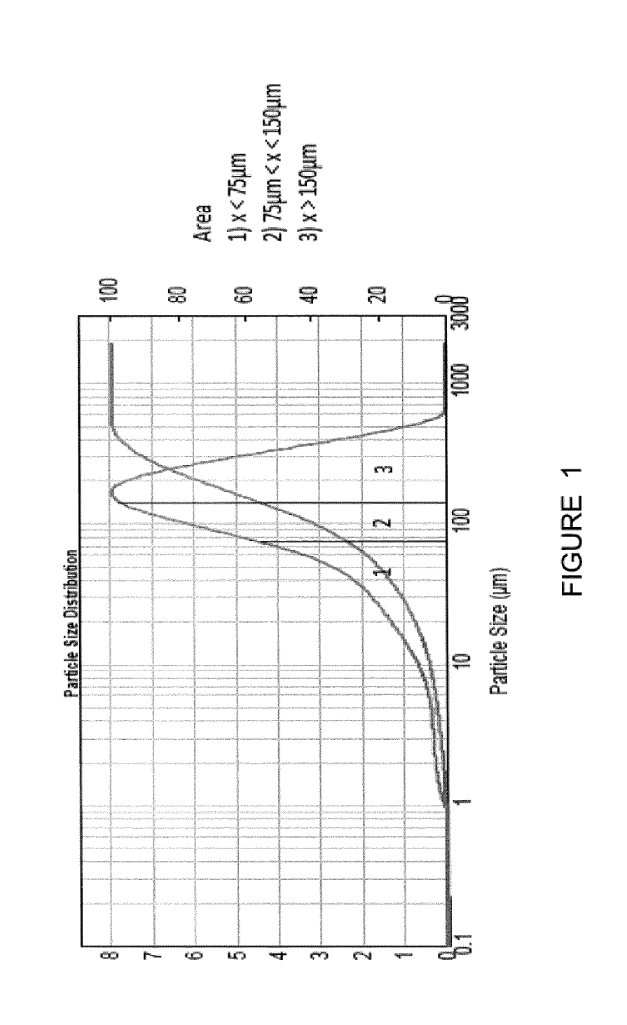

FIG. 2 presents photographs showing that the powder particles remain intact through the chemical reaction (FIG. 2A before reaction, FIG. 2B after reaction) although there is some fragmentation from the edges of the particles caused by the mechanical action of the washing process.

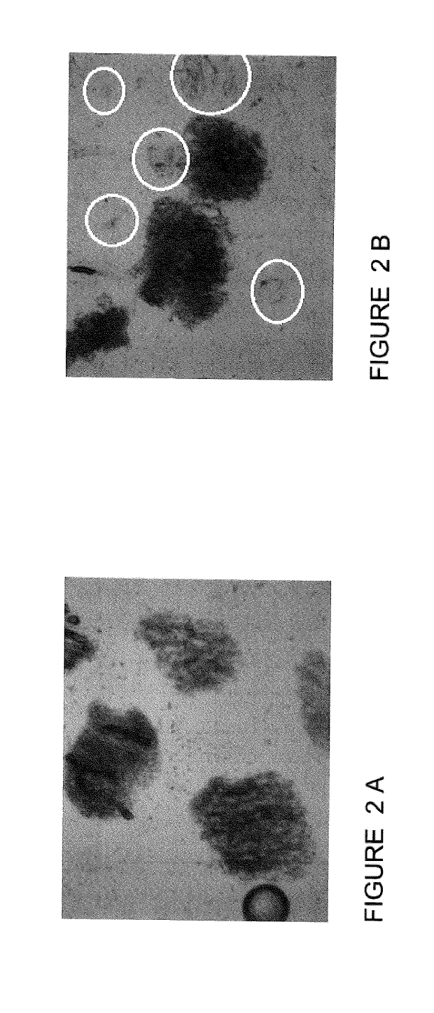

### DETAILED DESCRIPTION OF THE INVENTION

The process of the present invention is now described in further detail.

Optionally, the process of the present invention can be carried out as a continuous process, rather than being conducted batch-wise. This has significant advantages in terms of the efficiency of the process. The low viscosity of the mixture formed in the present invention enables continuous processing to be conducted without difficulty.

Plant Material

The starting material for the process of the present invention comprises an herbaceous plant material. The term “herbaceous” as defined herein refers to plants which are annual, biennial or perennial vascular plants, and is also used to refer to mosses, charophycean green algae and macro algae (brown seaweed). Whilst mosses, charophycean green algae and macro algae are not generally understood as being “herbaceous”, for convenience these plants are also referenced within the term “herbaceous” as used herein. In annual, biennial or perennial vascular plants, the stem matter dies after each season of growth when the plant becomes dormant (i.e. biennial or perennial plants) or dies (i.e. annual plants). Biennial or perennial plants survive unfavourable conditions underground and will regrow in more favourable conditions from such underground portions of the plant, typically stem, roots, or storage organs such as tubers. In contrast, the stems of woody species remain during any period of dormancy, and in a period of further growth will form growth rings which expand the girth of existing tissue.

Herbaceous plants are characterised by parenchymal tissue having an abundance of primary cell walls within the tissue. One skilled in the art would also be aware that the mosses, charophycean green algae and macro algae also consist of an abundance of primary cell walls (and hence are included within the term “herbaceous plant material” as used herein). Herbaceous plant material is preferably used as a starting material within the present invention. Optionally, the starting material of the present invention substantially consists of herbaceous plant material. It can be advantageous for the starting material of the present invention to consist of herbaceous plant material, and thereby exclude wood or wood products. Depending upon the intended end use of the cellulose-containing material, however, it may not, however, be necessary to totally avoid inclusion of non-herbaceous plant material (such as wood) within the plant starting material.

In particular, the plant material used in the process of the present invention can conveniently include vegetables, for example root vegetables, and fruit. Non-limiting examples of suitable root vegetables include carrot, sugar beet (also commonly referenced as “beet”), turnip, parsnip and swede. Exemplary fruit materials which can be used within the present invention includes apples, pears, citrus and grapes. Optionally, the plant material may be from tubers, for example potato. Sweet potato, yam, rutabaya and yucca root can also be used. Generally, it is anticipated that the process of the invention will be operated using waste or coproducts from the plant material after a main product has been extracted, for example sugarbeet pellets, vegetable peelings or citrus waste after juicing, jam-making or the like. However this is not strictly necessary and the process could be operated using vegetable or fruit grown specifically for that purpose. It is also not necessary for the plant material to be used as a starting material in the process of the present invention to comprise material from only one specific plant source. Optionally, a mixture of materials from different plant sources can be used. For example, the starting material can comprise a mixture of different root vegetables, a mixture of different fruits, a combination of fruit and vegetable(s), including a mixture of root vegetables together with a mixture of fruits.

Generally, the plant material to be used as a starting material for the present invention will not comprise a significant quantity of lignin. Optionally, the starting material for the present invention will comprise less than about 20 wt % lignin, for example less than about 10 wt % lignin, for example less than about 5 wt % lignin, for example less than 2 wt % lignin, for example less than about 1 wt % lignin. A number of methods for the measurement of lignin content are known in the art and include methods such as the Klason method, the acetyl bromide method and the thioglycolic acid method. Hatfield and Fukushima (Crop Sci. 45:832-839, 2005) discuss methods of lignin measurement.

The plant material can be raw plant material, i.e. uncooked. It is however desirable that the plant material has been washed, for example to remove any non-plant material debris or contaminants. The particles of plant material can advantageously be dry particles. By “dry” we mean that the particles of plant material contain less than 30 wt % water, for example contain less than 20 wt % water, for example contain less than 15 wt % water. Water is of course naturally present as part of the plant cell wall so even apparently very desiccated material may include some water content.

Production of the Particles

The particles can be formed using any suitable means. Preferably, water or other liquid is not added to the plant material prior to comminution to form the particles. Thus, the plant material is not in the form of a slurry or suspension during the comminution step. Thus the process can include a step of comminuting plant material in the absence of liquid to form particles of plant material. Optionally, the plant material contains less than 30 wt % water prior to comminution, for example contains less than 20 wt % water, for example contains less than 15 wt % water. In some embodiments, the plant material can be dried (e.g. at ambient temperature or at higher temperatures) before being formed into particles. The comminuted material can be screened to select particles of the desired size.

The particles of plant material can be formed by grinding or milling. For example, the plant material can be processed in a mill or using a grinding apparatus such as a classifier mill to provide particles of the required diameter size.

Preferably, a combination of a mechanically acting mill, i.e. one where the plant materials is crushed and turn apart and thus comminuted between actors, and a subsequent particle sizing is employed, e.g. by gravity or density, or sieving. However, the apparatus used to produce the particles from the plant material is not particularly critical to the successful operation of the process.

Particle Size

The particles of plant material used within the process of the present invention have a mean average diameter of from 10 μm to 800 μm. The term “diameter” refers to the measurement across the particle from one side to the other side. One skilled in the art would recognise the particles would not be perfectly spherical, but may be near-spherical, ellipsoid, disc-shaped, or even of irregular shape. One skilled in the art would also be aware that a range of diameters would be present within the starting material. To obtain the benefits of the present invention, it is not necessary to meticulously exclude very small quantities of particles which fall outside the stated particle diameter size. However, inclusion of particles of different diameter sizes within the starting material can, in some circumstances, adversely affect the quality of the end product.

Optionally, at least 60% by volume of the particles have a diameter of from 10 μm to 800 μm, for example at least 70% by volume of the particles have a diameter of from 10 μm to 800 μm, or at least 80% by volume of the particles have a diameter of from 10 μm to 800 μm, or at least 85% by volume of the particles have a diameter of from 10 μm to 800 μm, or at least 90% by volume of the particles have a diameter of from 10 μm to 800 μm, or at least 95% by volume of the particles have a diameter of from 10 μm to 800 μm, or even at least 98% by volume of the particles have a diameter of from 10 μm to 800 μm. Conveniently 99% by volume of the particles have a diameter of from 10 μm to 800 μm. In some circumstances it may be advantageous to ensure that substantially all of the particles have a diameter of from 10 μm to 800 μm.

Depending upon the source of the starting material and/or the intended end use of the cellulose-containing material, it can be advantageous to select particles having a mean average particle diameter size within a narrower range. For example, particles of plant material used within step (i) of the process of the present invention can have a mean average diameter of from 50 μm to 600 μm. In some circumstances, at least 60% by volume of the particles have a diameter of from 50 μm to 600 μm, for example at least 70% by volume of the particles have a diameter of from 50 μm to 600 μm, or at least 80% by volume of the particles have a diameter of from 50 μm to 600 μm, or at least 85% by volume of the particles have a diameter of from 50 μm to 600 μm, or at least 90% by volume of the particles have a diameter of from 50 μm to 600 μm, or at least 95% by volume of the particles have a diameter of from 50 μm to 600 μm, or even at least 98% by volume of the particles have a diameter of from 50 μm to 600 μm. Conveniently, 99% by volume of the particles have a diameter of from 50 μm to 600 μm. In some circumstances it may be advantageous to ensure that substantially all of the particles have a diameter size of from 50 μm to 600 μm. Alternatively, the particles of plant material used within step (i) of the process of the present invention can have a mean average diameter of from 250 μm to 550 μm. In some circumstances, at least 60% by volume of the particles have a diameter of from 250 μm to 550 μm, for example at least 70% by volume of the particles have a diameter of from 250 μm to 550 μm, or at least 80% by volume of the particles have a diameter of from 250 μm to 550 μm, or at least 85% by volume of the particles have a diameter of from 250 μm to 550 μm, or at least 90% by volume of the particles have a diameter of from 250 μm to 550 μm, or at least 95% by volume of the particles have a diameter of from 250 μm to 550 μm, or even at least 98% by volume of the particles have a diameter of from 250 μm to 550 μm. Conveniently 99% by volume of the particles have a diameter of from 250 μm to 550 μm. In some circumstances it may be advantageous to ensure that substantially all of the particles have a diameter of from 250 μm to 550 μm.

Alternatively, the particles of plant material used within step (i) of the process of the present invention can have a mean average diameter of from 300 μm to 550 μm. In some circumstances, at least 60% by volume of the particles have a diameter of from 300 μm to 550 μm, for example at least 70% by volume of the particles have a diameter of from 300 μm to 550 μm, or at least 80% by volume of the particles have a diameter of from 300 μm to 550 μm, or at least 85% by volume of the particles have a diameter of from 300 μm to 550 μm, or at least 90% by volume of the particles have a diameter of from 300 μm to 550 μm, or at least 95% by volume of the particles have a diameter of from 300 μm to 550 μm, or even at least 98% by volume of the particles have a diameter of from 300 μm to 550 μm.

Conveniently 99% by volume of the particles have a diameter of from 300 μm to 550 μm. In some circumstances it may be advantageous to ensure that substantially all of the particles have a diameter of from 300 μm to 550 μm.

Alternatively, the particles of plant material used within the process of the present invention can have a mean average diameter of from 50 μm to 200 μm. In some circumstances, at least 60% by volume of the particles have a diameter of from 50 μm to 200 μm, for example at least 70% by volume of the particles have a diameter of from 50 μm to 200 μm, or at least 80% by volume of the particles have a diameter of from 50 μm to 200 μm, or at least 85% by volume of the particles have a diameter of from 50 μm to 200 μm, or at least 90% by volume of the particles have a diameter of from 50 μm to 200 μm, or at least 95% by volume of the particles have a diameter of from 50 μm to 200 μm, or even at least 98% by volume of the particles have a diameter of from 50 μm to 200 μm. Conveniently 99% by volume of the particles have a diameter of from 50 μm to 200 μm. In some circumstances it may be advantageous to ensure that substantially all of the particles have a diameter of from 50 μm to 200 μm.

Alternatively, the particles of plant material used within the process of the present invention can have a mean average diameter of from 50 μm to 100 μm. In some circumstances, at least 60% by volume of the particles have a diameter of from 50 μm to 100 μm, for example at least 70% by volume of the particles have a diameter of from 50 μm to 100 μm, or at least 80% by volume of the particles have a diameter of from 50 μm to 100 μm, or at least 85% by volume of the particles have a diameter of from 50 μm to 100 μm, or at least 90% by volume of the particles have a diameter of from 50 μm to 100 μm, or at least 95% by volume of the particles have a diameter of from 50 μm to 100 μm, or even at least 98% by volume of the particles have a diameter of from 50 μm to 100 μm. Conveniently 99% by volume of the particles have a diameter of from 50 μm to 100 μm. In some circumstances it may be advantageous to ensure that substantially all of the particles have a diameter of from 50 μm to 100 μm.

Alternatively, the particles of plant material used within the process of the present invention can have a mean average diameter of from 100 μm to 300 μm. In some circumstances, at least 60% by volume of the particles have a diameter of from 100 μm to 300 μm, for example at least 70% by volume of the particles have a diameter of from 100 μm to 300 μm, or at least 80% by volume of the particles have a diameter of from 100 μm to 300 μm, or at least 85% by volume of the particles have a diameter of from 100 μm to 300 μm, or at least 90% by volume of the particles have a diameter of from 100 μm to 300 μm, or at least 95% by volume of the particles have a diameter of from 100 μm to 300 μm, or even at least 98% by volume of the particles have a diameter of from 100 μm to 300 μm. Conveniently 99% by volume of the particles have a diameter of from 100 μm to 300 μm. In some circumstances it may be advantageous to ensure that substantially all of the particles have a diameter of from 100 μm to 300 μm.

Optionally, the particle size distribution of the particles used in step (i) can be 500 μm or less, for example 400 μm or less, 300 μm or less, 200 μm and less or even 100 μm or less. The “particle size distribution” refers to the degree of variation in particle diameter size within the material sample. For example, where the starting material consists of particles having a diameter of from 200 μm to 500 μm, the particle size distribution is 300 μm. Likewise, where the starting material consists of particles having diameters of from 50 μm to 250 μm, the particle size distribution is 200 μm. It is recognised that to obtain a powder in which 100% of the particles fall within the selected particle size distribution is practically impossible, and accordingly the reference to a powder having a specified particle size distribution means that at 95% by volume (preferable 98% by volume, 99% by volume, 99.5% by volume or even 99.9% by volume) of the particles fall within that distribution. Particles of the required diameter and within the predetermined particle size distribution can be selected using known methods, including (but not limited to) sieving the particle mixture with one or more sieves of known sieve size. For example, passing the material sample through a sieve having a mesh size of 500 μm will only allow particles having a particle diameter of 500 μm of less to pass through. The sieved material can then be sieved again using a sieve having a smaller mesh size, for example a mesh size of 300 μm. The particles retained on the smaller mesh (i.e. which do not pass through) will have a particle size distribution of 200 μm and range in size from 300 μm to 500 μm. Of course, sieves of alternative sieve size and in different combinations can be used to obtain any required particles diameter size range and particle size distribution. Alternatively, a classifier mill or other suitable means can be used to select particles of the required particle size and size distribution.

Step (i): Step (i) of the process involves contacting the particles of plant material with a peroxide reagent and water. It is not essential for the peroxide reagent to be added simultaneously with the water. However, it is often convenient to add the water and peroxide reagent simultaneously. For example, it is possible to premix the peroxide reagent with the water and then to add the water-peroxide reagent mixture to the plant material particles. Alternatively, it is possible to add water to the particles of plant material to form an aqueous slurry, and then to add the peroxide reagent to the slurry. Advantageously, addition of the water and/or peroxide reagent is accompanied by stirring of the resultant mixture to facilitate formation of a homogenous composition.

The volume of water to be added is not particularly critical, but may typically be from 2 litres to 30 litres water per kg plant material particles. This is in addition to any solution of peroxide reagent which may additionally be added. One of the benefits of the present invention is the relatively high percentage of solids which can be present within the mixture after the addition of water and peroxide reagent. In some embodiments, the mixture formed in step (i) can contain more than 2 wt % solids (which is the level typically achieved using prior art processes such as those described in WO 2014/147392 and WO 2014/147393). In some embodiments, the mixture formed in step (i) can contain at least 3 wt % solids, for example at least 4 wt % solids or at least 5 wt % solids.

Peroxide Reagent: The peroxide reagent breaks down the particles of plant material and aids in release of the cellulose-containing material end product. The peroxide reagent can be an organic peroxide or an inorganic peroxide. Exemplary organic peroxides include peroxycarboxylicacids (such as peracetic acid and peroxybenzoicacids, e.g. m-chloroperoxybenzoicacid) and hydroperoxides, including alkyl hydroperoxidesand acyl hydroperoxides (such as benzoylperoxide). Exemplary inorganic peroxides include acid peroxides (such as peroxysulphuricacid and peroxyphosphoric acid) and peroxides of the alkali and alkaline earth metal peroxides (such as sodium peroxide and barium peroxide). Hydrogen peroxide is preferred.

In one embodiment, hydrogen peroxide in a concentration of 35% (w/w in water) is added in a ratio of from 0.1:1 to 0.5:1 of peroxide: plant solids. Although a catalyst is not essential for the process of the present invention, it may be desirable in some circumstances to include a catalyst for the peroxide reaction step. Suitable catalysts include transition metal catalysts, for example manganese catalysts. However, the process of the present invention will generally be conducted without the requirement for a catalyst.

Step (ii): In step (ii) the aqueous mixture produced in step (i) is allowed to hydrate for a period of time sufficient until the pH of the mixture is measured to be pH 4.5 or lower, optionally less than pH 4.5. Immediately after addition of the water and peroxide reagent, the pH of mixture as measured at this point is significantly higher, typically approximately pH 6 to pH 7. The period of time required to reach the required degree of hydration (as determined by an endpoint pH of 4.5 or lower) can vary with parameters such as: particle size, temperature (both ambient temperature and/or the temperature of the slurry), concentration of peroxide reagent and the like. It has been noted that the hydration step proceeds more quickly with increased temperature and it may be beneficial to pre-heat the water (for example to temperatures of from 30 to 100° C., for example 60 to 90° C.) prior to its addition to the particulate plant material.

As noted above, the end point pH is 4.5 or lower, optionally is less than 4.5, for example is 4.4 or less, 4.3 or less, 4.2 or less, 4.1 or less, 4.0 or less, 3.9 or less, 3.8 or less, 3.7 or less, 3.6 or less, or 3.5 or less.

Optionally the end point pH is from 3.0 to 4.5, for example is 3.0 to 4.4, for example is 3.0 to 4.3, for example is 3.0 to 4.2, for example is 3.0 to 4.1, for example is 3.0 to 4.0, or for example is 3.0 to 3.5.

Optionally the end point pH is from 3.5 to 4.5, for example is 3.5 to 4.4, for example is 3.5 to 4.3, for example is 3.5 to 4.2, for example is 3.5 to 4.1, or for example is 3.5 to 4.0.

Optionally, the mixture formed in step (ii) can be heated for part or all of the time needed to reach the end point pH. Heating can be advantageously accompanied by gentle stirring or agitation of the mixture to ensure that the temperature is reasonably consistent throughout the whole mixture volume, such as in conventional reaction vessels. Suitable agitation can be achieved by causing the mixture to flow along a pipe or other conduit.

Heating can be conducted by any suitable means, but conveniently can be carried out by passing the mixture through a pipe which has a heating apparatus around its external circumference. Suitable heating apparatuses include conventional thermal heating elements and/or a microwave apparatus which is focused onto the pipe interior. Optionally, the mixture is heated to a temperature of from 30 to 110° C., for example 90 to 95° C.

In some embodiments however, it is not necessary for the mixture to be heated beyond ambient (e.g. 15 to 25° C.) and this has clear benefits in reducing the cost of producing the cellulose-containing material.

The time taken to reach the required end point pH may vary depending upon conditions such as particle size, temperature, degree of agitation (stirring) of the mixture and the like. Typically the reaction time will be around 2 to 6 hours, for example may be 3 to 4.5 hours.

The viscosity of the mixture formed at the start of step (i), i.e. immediately after introduction of the water or the water-peroxide reagent mixture, will depend upon factors such as the starting material used and the solids level within the mixture, but a typical value at 1% solids will be approximately 5 to 30 cPs. Once the end point pH has been reached, the viscosity of the mixture has generally increased but is still relatively low. Again the exact value obtain will depend upon the starting material, reaction conditions etc., but a typical value at 1% solids is approximately 30 to 200 cPs. During step (ii) the particles of plant material become hydrated and swell, increasing in size. Thus, as an example, a 100 μm particle as used in the starting material of step (i) can swell to have a diameter of approximately 130 μm by the end of step (ii).

Optionally step (ii) can include: having a pH of pH 4.5 or less (for example pH 3.0 to 4.5); and (iia) washing or neutralising the hydrated mixture to form a treated hydrated mixture.

Optionally step (ii) can include: allowing the mixture from step (i) to hydrate to form an hydrated mixture having a pH of pH 4.5 or less (for example pH 3.0 to 4.5); (iia) washing or neutralising the hydrated mixture to form a treated hydrated mixture; and (iib) bleaching the treated hydrated mixture from step (iia) to form a bleached hydrated mixture.

Optionally step (ii) can include: allowing the mixture from step (i) to hydrate to form an hydrated mixture having a pH of pH 4.5 or less (for example pH 3.0 to 4.5); (iia) washing or neutralising the hydrated mixture to form a treated hydrated mixture; (iib) bleaching the treated hydrated mixture from step (iia) to form a bleached hydrated mixture; and (iic) washing the bleached hydrated mixture of step (iib).

As indicated above, step (ii) can optionally include one or more washing steps (i.e. steps (iia) and (iic)). One of the major benefits of the process of the present invention is the ease with which washing of the material can be achieved, despite having a higher wt % solids compared to prior art processes. Typically, washing requires the cellulose material to be separated from the liquid fraction, and then re-suspended (optionally with agitation or stirring) in clean liquid, such as water. The washing step removes any excess peroxide reagent and/or bleach, and also any soluble by-products formed in step (i).

An alternative to the washing step of step (iia) is to neutralise the hydrated mixture so that the pH is changed to pH 6 to 8, preferably to pH 6.5 to 7.5, i.e. to be pH at or close to pH 7. Neutralising the mixture of step (ii) after the end point pH has been reached can reduced or even eliminate the requirement for a washing step, thereby reducing the amount of water consumed during the manufacturing process, which is an important environment consideration. Neutralisation can be achieved by addition of an appropriate amount of a base or of a buffer sufficient to change the pH of the mixture to pH 6 to 8. The base or buffer can be added in any convenient form, but typically will be added as a powder or in the form of an aqueous solution. Alkalis such as sodium hydroxide, potassium hydroxide, calcium carbonate or the like can conveniently be used. Optionally, once the hydrated mixture has been neutralized (and optionally mixed therewith}, the cellulose-containing material can separated from the liquid fraction by any suitable means before being re-suspended in a suitable volume of water. Alternatively, the step of neutralisation can be performed after the cellulose-containing material has been separated from the liquid fraction. For example, the cellulose-containing material can be separated and then re-suspended before a suitable amount or alkali is added. Alternatively, the separated cellulose-containing material can simply be re-suspended in an alkaline solution.

The step of separating the cellulose-containing material from the liquid fraction can be achieved using any suitable apparatus or process, including without limitation filtration (simple or vacuum filtration), centrifugation, membrane filtration etc. A woven filter can be used. Alternatively a mesh filter can be used. Optionally, where filtration is used during the washing step, the filter has a pore size of 200 μm or less, for example has a pore size of 100 μm to 200 μm. A smaller pore size can also be used.

Optionally, the washing or neutralising step (iia), if present, is conducted in a manner which is compatible with a continuous manufacturing process. For example a filter at an angle of approximately 45° to the horizontal can be used, with the material to be filtered being dropped onto the filter from above so that liquid drains through the filter whilst solids are retained on the upper surface of the filter. The angle of the filter cause these retained solids to slide gently down the filter's upper surface onto a belt, or into a hopper or other receptacle ready for further processing. Alternatively a belt filter press can be used.

Step (iia) can advantageously be conducted as soon as the end point pH has been reached. Where step (iia) is a washing step, washing by separation and re-suspension can be repeated more than once, if required. Alternatively, where the step (iia) is a neutralising step, the base or buffer can simply be added to the mixture as soon as the end point pH has been reached. The bleaching step of step (iib) can be conducted using an oxidant. A suitable oxidant is sodium hypochlorite. The oxidant can, for example, be added at a concentration of 10 to 40% (v/v water), for example 35% (v/v water) in a ratio of 5:1 to 1:1, for example 2:1 oxidant to plant solids. Step (iib) can be conducted at ambient temperature. Alternatively some heat can be applied to the mixture, for example temperatures of up to 60° C. can be used. Typically the oxidant is added and the mixture is gently stirred or other agitated for a suitable period of time. The oxidant reduces the coloration of the material, rendering it more acceptable for certain applications, for example as an additive for paint or for use in composite materials. Generally step (iib) is carried out for a period of 30 minutes of less, for example 20 minutes or less, or even 10 minutes or less, such as 5 to 10 minutes.

The action of the oxidant has been noted to take place very quickly, so that addition of the oxidant for a very short period (such as 2 minutes or less) can be sufficient.

As noted above, the bleaching step (iib) can be followed by a further washing step (iic) which can be carried out as described above for step (iia). Where both steps (iia) and (iic) are present (and where step (iia) is also a washing step), it is not necessary for both steps to be carried out in the same way or to the same specification.

Step (iii): Once step (ii) is complete (including any optional washing, neutralising and/or bleaching steps), the hydrated material is subjected to a homogenisation step. At this stage of the process, a rapid increase in viscosity of the material is obtained.

For example a viscosity of 5000 cPs can be obtained, for example a viscosity of 4000 cPs, for example a viscosity of 3500 cPs, for example a viscosity of 3000 cPs, for example a viscosity of 2500 cPs, for example a viscosity of 2000 cPs. The viscosity required can be determined by controlling the extent of homogenization performed. The homogenisation can alternatively be conducted until the required particle size is obtained. Generally a particle size of from 75 to 500 μm is suitable for most applications.

Optionally step (iii) can include: (iiia) homogenising the mixture from step (ii) to form an homogenised mixture;

(iiib) washing the homogenised mixture to form a washed homogenised mixture; and

(iiic) isolating the cellulose-containing material. As noted above, the homogenising step (iiia) can be followed by a further washing step (iiib) which can be carried out as described above for the washing step(s) of step (ii). Where step (iiib) is present, it is not necessary for this step to be carried out in the same way or to the same specification as either of step (iia) or (iic). Step (iiic) refers to a step of isolating the cellulose-containing material. This can be achieved, for example, by a final filtration step which may also include a step of concentrating the cellulose-containing material, for example by removing further liquid from the material in order to form it into a paste, cake or other more concentrated form. Optionally, the cellulose-containing material contains at least 5 wt % solids, for example at least 10 wt % solids, for example 15 wt % solids, for example 20 wt % solids, for example 25 wt % solids, for example 30 wt % solids. A belt filter press can be used to achieve the more concentrated form of material. At levels of over 15 wt % solids the material can be pelletized, for example can be grated or can be extruded into strings or other shapes.

Uses of the Cellulose-Containing Material: The cellulose-containing material can be used as an additive in a wide range of different industries including (without limitation) food and drink applications, personal care products, paint systems, concretes, drilling muds, composite materials such as epoxies and the like. The cellulose-containing material has useful viscosity-adjusting properties and can be used to improve the rheology of products. The cellulose-containing material is also useful as a mechanical enhancer for example to increase the scrub resistance of a coating. It is also useful as an anti-cracking agent, particularly for paints and concretes.

Typically, the cellulose-containing material formed in the process of the present invention need only be added in surprisingly small quantities to achieve a different effect of the physical properties of the material into which it has been incorporated. For example the cellulose-containing material formed in the process of the present invention need only be added in an amount of from 10 wt %, for example 8 wt %, for example 5 wt %, for example 3 wt %, for example 2 wt % or even 1 wt % or even less. In some applications the cellulose-containing material formed in the process of the present invention need only be added in an amount of 0.5 wt % or less.

The material to which the cellulose-containing material is added may be and aqueous based system (for example a solution, suspension or dispersion). Mention I may be made of water-based paints as being of particular interest. In paint and plaster applications the cellulose-containing material facilitates even drying and thus prevents the development of micro and macro-cracking.

Also of relevance are food products and drink products. Food products where rheological modification may be of benefit include any product which is processed in the form of a slurry, suspension or liquid. Thus the cellulose-containing material can beneficially be added to dairy products (milk products, yoghurts, creams, custards, ice creams or other frozen desserts, and the like), to processed fruits (in the form of smoothies, pie fillings, jams or sauces) and to sauces, gravies, mayonnaise etc. The cellulose-containing material may be of particular benefit in baked products, in particular in gluten-free products such as gluten free breads, cakes and biscuits. Additionally, the cellulose-containing material of the present invention can be useful to at least partially replace fats in high fat foodstuffs (e.g. in chocolate, puddings and desserts) by providing a smoother mouthfeel with a lower fat content than would otherwise be acceptable, and/or to increase the dietary fibre content of selected foodstuffs, for example in products formed using refined flours such as pasta, noodles, breads, biscuits, cakes and pastry products.

The cellulose-containing material formed in the process of the present invention can also be used in paper, cardboard and packaging manufacture. Small quantities of the cellulose-containing material (for example 10 wt % or less) can be added in order to provide increased stiffness and tear strength thereby allowing thinner quantities of materials to be used.

The cellulose-containing material formed in the process of the present invention can also be used in paints and plasters. In paints, it was found that the presence of the material in comparatively low concentrations allowed to increase the open time of coating films, while also promoting flow, but also reducing drying times and final coating properties. This not only increases the useful time for applying films, but also allows for better surface properties such as increased gloss and reduced pin-holing. At the same time, more of the latent solvents present in the films, in particular water, can evaporate, and hence the actual drying time is advantageously reduced.

A further surprising effect of the presence of the cellulose-containing material formed in the process is the increase of opacity in pigmented films, permitting a reduction in the amount of pigments, such as TiO₂, needed.

Likewise, the cellulose-containing material of the present invention can enhance the mechanical properties of recycled paper. The cellulose-containing material can also be used as part of a coating to enhance the visual appearance of the paper or cardboard.

Further, the cellulose-containing material formed in the process of the present invention can also be used in personal care products, including soaps, shampoos, shower, bath and body gels as well as in such products such as skin creams, lotions and cosmetics where it can enhance the rheology of the product.

Additionally, the product also finds utility in medical based creams, ointments, lotions and the like. The product of the present invention further has the advantage of being of natural origin.

Preferred or alternative features of each aspect or embodiment of the invention apply mutatis mutandis to each aspect or embodiment of the invention (unless the context demands otherwise).

The term “comprising” as used herein means consisting of, consisting essentially of, or including and each use of the word “comprising” or “comprises” can be independently revised by replacement with the term “includes”, “consists essentially of” or “consists of”.

All documents referred to herein are incorporated by reference. Any modifications and/or variations to described embodiments that would be apparent to one of skill in art are hereby encompassed. Whilst the invention has been described herein with reference to certain specific embodiments and examples, it should be understood that the invention is not intended to be unduly limited to these specific embodiments or examples.

The present invention is now further described with reference to the following non-limiting examples.

### EXAMPLES

### Comparative Example

900 g of sugar beet pellets were washed and hydrated by adding them to warm water, with dirty water being drained through a sieve. This sugar beet hydrate is placed in a large bucket in excess water and agitated before being scooped out with a colander and washed with water, to ensure that no stones/grit enter the next stage of processing.

The washed sugar beet is then cooked for 3 hours at 100° C., before being homogenised using a Silverson FX homogeniser fitted with initially coarse stator screens and moving down to the small holed emulsifier screen (15 min process time for each screen). The solids are measured using an Oxford solids meter and the mixture adjusted to 2% solids by addition of clean water.

A sample of the mix is then placed in a 5 litre glass reaction vessel. Peroxide based on a ratio of aqueous peroxide solution (at 35% w/w in water) to the dry solids of 0.5:1 is added when the mix is heating. The temperature is maintained for 2 hours at 90° C. (once it reaches 90° C.), by which time the pH has dropped from around 5 to 3.5.

The reaction liquid is then removed from the vessel and washed prior to bleaching. Washing was achieved by mixing the reaction mixture with clean water and then passing through a filter, then re-suspending the solids captured in the sieve in more clean water and re-filtering.

Bleaching is then carried out by re-suspending the washed material in clean water and placing it back in the vessel. Bleaching is performed at 60° C., with a 2:1 bleach (2 parts of bleach solution with 10% active chlorine to 1 part solids, for 30 minutes). The material is then washed, as previously described and homogenised for 30 minutes on the fine slotted stator screen of the Silverson FX homogeniser. The material is then drained through a filter and pressed between absorbent cloths to a desired final solids content.

Method: Dried sugar beet pellets were ground into powder using a flour mill, and the particle diameter was determined. The sugar beet was then subjected to a hydrogen peroxide reaction in water. All hydrogen peroxide reactions were carried out in a 5 L glass reactor with a total reaction mixture volume of 4000 ml. Water (3879 ml) in the reactor was heated to 90° C. and hydrogen peroxide (40 g) was added. Sugar beet powder (89 g, 89% solids, particle diameter size of either A: 75-150 μm or B: 150 μm and above) was added directly to peroxide water mixture. After the required pH drop or reaction time the reaction was quenched by pouring the mixture through a filter mesh with 152 μm diameter holes. Samples were filtered using the mesh filter, by mixing the reaction liquid with clean water and pouring this onto a filter screen. The paste was then removed from the filter, clean water was added and then the new mixture poured back over the mesh filter. This process was repeated as required to ensure good washing.

After the hydrogen peroxide level in the washed paste had dropped to less than 1 ppm, the bleach reaction was carried out by diluting the washed paste to 0.5% solids. The diluted mixture was heated to 60° C. and bleach was then added in an amount of 2:1 ratio to solids. The same filter process was applied as had been conducted after the peroxide stage and the resultant clean paste was prepared for homogenisation.

Homogenisation was carried out at 0.5% solids with a benchtop Silverson homogeniser. The volume of the homogenised solution was around 4000 ml, (adjusted as necessary to always ensure 0.5% solids). After 30 min at 7500 rpm the smooth suspension was poured gently into a filter cloth and left to drain until the solids were greater than 1%. A pre-press viscosity was taken and the sample was then pressed between absorbent cloths in a hydraulic press.

Example 1: The method as described above was conducted using sugar beet powder having a particle diameter size ranging up to 700 μm, although 99.55% (by volume) of the particles had a diameter size of 500 μm or less. The reaction time was 4 hours 15 minutes and the pH of the mixture at the end of the peroxide reaction was 3.30. The reacted mixture was filtered using a cloth filter. Samples of the mixture were taken at each stage of the process and the viscosity of each of these samples was measured using a Brookfield viscometer with spindle rotated at 10 rpm at 20° C.

Table 1 shows the viscosity of the powder mixture compared to the comparative example process commencing from sugar beet pellets, at various stages through the process up to the end of the bleach reaction.

TABLE 1
Comparison of Viscosity Values (in cPs) during the Comparative
Example and during the process of Example 1

| Sample | Spin speed | 1 | 2 | 3 |
|---|---|---|---|---|
| Comparative | 100 rpm | 63.6 | 336 | 138 |
| | 50 rpm | 73.6 | 612 | 186 |
| | 20 rpm | 104 | 1265 | 376 |
| | 10 rpm | 140 | 2210 | 652 |
| Example 1 | 100 rpm | 8 | 39 | 49 |
| | 50 rpm | 10 | 56 | 74 |
| | 20 rpm | 15 | 100 | 135 |
| | 10 rpm | 20 | 160 | 240 |

Process Stage:

1. Start of hydrogen peroxide reaction, 2% solids, 70° C. Comparative example/60° C. Example 1.
2. After washing following hydrogen peroxide reaction, 1% solids, 20° C.
3. Bleach reaction, but at 1.5% solids, 56° C. Comparative example/30° C. Example 1.

The process of the invention provides significantly lower viscosity throughout the process up until the end of the bleach reaction after which the final homogenisation greatly increased the viscosity of the powder material and became the same as that for the comparative process using cooked and homogenised pellets. The powder process therefore enables a higher solids content to be used throughout the process which significantly increases efficiency.

The process of Example 1 was repeated, but using higher concentrations of solids during the hydrogen peroxide reaction step (i.e. process stage 3 in Table 1). The following viscosity measurements were obtained. All other process parameters were kept as in Example 1. The results are shown in Table 2.

TABLE 2
Viscosity Values (in cPs) using increased solids concentrations in
Hydrogen Peroxide Reaction

| | 4% solids, start of | 6% solids, start of | 6% solids end of |
|---|---|---|---|
| Spin Speed | reaction | reaction | reaction |
| 100 rpm | 13 | 2840 | 396 |
| 50 rpm | 10 | 4400 | 622 |
| 20 rpm | 10 | 7450 | 1030 |
| 10 rpm | 20 | 12200 | 990 |

The conventional process could not be run at these higher solids content because it was not possible to stir the material during the reactions due to its extremely high viscosity. Thus 2% solids is the limit for the conventional process but it is possible to perform the reaction according to the invention at solids content which is 2 to 3 times higher than that possible using the conventional process.

Example 2: The process of Example 1 was repeated, but using powder having a particle diameter size ranging from 75 μm to 150 μm. The reaction time was 4 hours 30 minutes and the pH of the mixture at the end of the peroxide reaction was 3.43. The reacted mixture was filtered using a mesh filter (pore size 152 μm). The final viscosity of the end product was 3780 cPs.

Example 3: Example 2 was repeated, but using powder having a particle diameter size ranging up to 700 μm, with 99.55% (by volume) of the particles having a diameter size of 500 μm or less. The reaction time was 4 hours 30 minutes and the pH of the mixture at the end of the peroxide reaction was 3.26. The reacted mixture was filtered using a mesh filter (pore size 152 μm). The final viscosity of the end product was 3370 cPs. Example 3 uses a relatively wide particle distribution (0-700 μm) when compared with Example 2 where the particle size distribution is 75-150 μm. The viscosity obtained using the narrow distribution of less than 100 μm in Example 2 was noticeably higher (3780 cP) compared to that of the wider particle distribution in Example 3 (3370 cP). Thus, a particle size distribution range of 100 μm or less can improve the viscosity obtained.

Example 4: The process of Example 3 was repeated, but included an additional initial step in which the powder was placed into hot water at 80° C. for 60 min to investigate whether pre-hydration of the powder would affect the peroxide reaction. After the hot water step, the process as described in Example 3 was followed. The time taken to reach pH 3.2 was reduced to 3 hours. The end viscosity was not changed significantly, being 3370 cPs compare to 3360 cPs for Example 3 without the pre-hydration step). Thus, the viscosity was not changed showing that a cooking/pre-hydration step is not required, in contrast to the prior art process.

Example 5: The method as described above in Example 1 was conducted using sugar beet powder with particle size greater than 150 μm (150-700). The reaction time was 3 hrs 30 min and the end pH was 3.4. The end viscosity was 3160 cPs showing that larger particles gave a slightly poorer end viscosity than the full particle range up to 700 μm and significantly poorer than particles between 75 and 150 μm. Samples of the mixture were taken at each stage of the process and the average particle size for each sample was measured as described above for Example 1. The results are set out in Table 3 and include normalized values calculated using the following equation:

$$n = \frac{m}{h}$$

Where n is the normalised value, m is the measured average in micrometres and h is the largest measured average in that experiment.

TABLE 3
Particle Size

| | Av. diameter | Normalized |
|---|---|---|
| Dry powder | 311 | 0.94 |
| Peroxide start | 332 | 1.00 |
| Peroxide end | 236 | 0.71 |
| 151Wash | 216 | 0.65 |
| Bleach start | 251 | 0.76 |
| Bleach end | 224 | 0.67 |
| 2nd Wash | 282 | 0.85 |
| Post homogenization | 79 | 0.24 |

Example 6: The method as described above in Example 2 was conducted using sugar beet powder with particle size less than 75 μm (i.e. particles size range of 0 to 75 μm). The reaction time was 3.5 hours and the end pH was 3.38. The end viscosity was 2260 cPs showing that very small particles gave a poorer end viscosity than the full particle range up to 700 μm and significantly poorer than particles between 75 and 150 μm.

Example 7: Swelling capacity without peroxide treatment: 30 g of dry untreated sugar beet powder was produced by grinding dry sugar beet material. All particles had a diameter size was below 800 μm. The untreated powder was hydrated in hot water for an hour then drained through filter mesh. The result was a paste weighing 235 g. Although the particles did not swell much they can incorporate water into their structure equal to many times their own weight. The swelling capacity of the powder in terms of weight increase is 683% but in terms of size increase it is less than 30%, demonstrating the ability of the particles to accommodate a significant quantity of fluid without a corresponding increase in size which would cause viscosity increase.

1. A process for preparing cellulose-containing material, the process comprising the steps of:
(i) contacting particles of plant material with a peroxide agent and water;
(ii) allowing the mixture from step (i) to hydrate until the pH of the mixture is pH 4.5 or less; and
(iii) homogenising the mixture from step (ii) and isolating the cellulose containing material,
and wherein the particles of plant material in step (i) have an average particle diameter of from 10 μm to 800 μm.

2. The process as claimed in claim 1, wherein the process comprises a step of comminuting plant material in the absence of liquid to form particles of plant material with an average particle diameter of from 100 μm to 800 μm.

3. The process as claimed in claim 1 wherein the particles of plant material have an average particle diameter of from 100 μm to 300 μm.

4. The process as claimed in claim 1 wherein the particles of plant material have a particle size distribution of from 100 μm to 200 μm.

5. The process as claimed in claim 1 wherein the particles of plant material have a particle size distribution of between 75 and 150 μm.

6. The process as claimed in claim 1 wherein the peroxide agent is hydrogen peroxide.

7. The process as claimed in claim 1 wherein the plant material substantially comprises a herbaceous plant material.

8. The process as claimed in claim 7 wherein the herbaceous plant material is a root vegetable, a tuber or a fruit.

9. The process as claimed in claim 8 wherein the root vegetable is carrot, sugar beet, turnip, parsnip or swede.

10. The process as claimed in claim 7 wherein the herbaceous plant material is sugar beet.

11. The process as claimed in claim 8 wherein the tuber is potato, sweet potato, yam, rutabaya or yucca root.

12. The process as claimed in claim 8 wherein the fruit is apple, pear, citrus or grape.

13. The process as claimed in claim 1 wherein the peroxide agent is added to the water to form a water-peroxide reagent mixture, and the water-peroxide reagent mixture is then added to the particles of plant material in step (i).

14. The process as claimed in claim 1 wherein in step (ii) the plant material is allowed to hydrate.

15. The process as claimed in claim 1 wherein step (ii) comprises: allowing the mixture from step (i) to hydrate to form a hydrated mixture having a pH of 4.5 or less; and washing or neutralising the hydrated mixture to form a treated hydrated mixture.

16. The process as claimed in claim 1 wherein step (ii) comprises: allowing the mixture from step (i) to hydrate to form a hydrated mixture having a pH of 4.5 or less; washing or neutralising the hydrated mixture to form a treated hydrated mixture; and bleaching the treated hydrated mixture to form a bleached hydrated mixture.

17. The process as claimed in claim 1 wherein step (ii) comprises: allowing the mixture from step (i) to hydrate to form an hydrated mixture having a pH of 4.5 or less; washing or neutralising the hydrated mixture to form a treated hydrated mixture; bleaching the treated hydrated mixture to form a bleached hydrated mixture; and washing the bleached hydrated mixture.

18. The process as claimed in claim 1 wherein the mixture from step (ii) has viscosity of 30 to 200 cPs.

19. The process as claimed in claim 1 wherein homogenising the mixture from step (ii) in step (iii) provides a mixture with a viscosity of 2000 to 5000 cPs.

20. The process as claimed in claim 1 which is carried out in a continuous manner.

21. The process as claimed in claim 1 wherein the cellulose-containing material has a particle size of from 75 to 500 μm.

22. A cellulose-containing material obtainable by the process of claim 1.

23. A paint or plaster composition comprising a cellulose-containing material according to claim 21.

24. A food and/or drink composition comprising a cellulose-containing material according to claim 21.

25. A personal care product comprising a cellulose-containing material according to claim 21.

26. A drilling mud composition comprising a cellulose-containing material according to claim 21.

27. A composite material composition comprising a cellulose-containing material according to claim 21.

28. Use of a cellulose-containing material according to claim 21 to increase the film open time of a paint or plaster composition.

29. Use of a cellulose-containing material according to claim 21 to increase the opacity of coatings.
